(19) United States
(12) Patent Application Publication
(10) Pub. No.: US 2024/0148938 A1
(43) Pub. Date: May 9, 2024

(54) METHOD FOR REALIZING CARTILAGE REGENERATION BY MEANS OF INOCULATING GEL CARTILAGE INTO FRAME STRUCTURE

(71) Applicant: SHANGHAI RESTHETIC BIO CO., LTD, Shanghai (CN)

(72) Inventors: Yu LIU, Shanghai (CN); Guangdong ZHOU, Shanghai (CN); Zheng CI, Shanghai (CN)

(21) Appl. No.: 18/262,216

(22) PCT Filed: Jan. 18, 2022
(86) PCT No.: PCT/CN2022/072584

§ 371 Date: Jan. 24, 2024

(30) Foreign Application Priority Data
Jan. 20, 2021 (CN) 202110077195.X

(51) Int. Cl. A61L 27/38; A61L 27/18; A61L 27/36; A61L 27/52; A61L 27/56

(52) U.S. Cl. CPC A61L 27/3817; A61L 27/18; A61L 27/3633; A61L 27/3852; A61L 27/52; A61L 27/56; A61L 2430/06

(57) ABSTRACT

A method for realizing cartilage regeneration involves inoculating a gel cartilage into a frame structure. Specifically, cartilage cells are prepared into a gel cartilage by means of amplification and dense inoculation; and the gel cartilage is inoculated into a decalcified bone matrix to prepare a gel cartilage—decalcified bone complex, so that cartilage regeneration is realized, and the gel cartilage—decalcified bone complex is used in various filling treatments and bone repair.

### TECHNICAL FIELD

The present invention relates to biomedical tissue engineering, in particular to a method for realizing cartilage regeneration by means of inoculating gel cartilage into frame structure.

### BACKGROUND

In recent years, with the rapid development of the economy and society, cartilage defects caused by various types of trauma and congenital malformations have become increasingly common. Most cartilage defects caused by trauma or congenital deformities are difficult to repair rely on patient self. At present, the treatment methods for articular cartilage defects in clinical practice are mainly palliative treatment and restorative treatment. Palliative treatment mainly includes arthroscopic debridement and chondroplasty. This type of treatment can clean the uneven cartilage surface of the joint surface and remove cartilage fragments, etc., to restore the smooth and flat joint surface. This method is less traumatic and can relieve the patient's symptoms to a certain extent, but its curative effect is limited and cannot effectively relieve the development of arthritis. Restorative treatment includes microfracture treatment, osteochondral transplantation, etc. Although this kind of treatment can repair focal articular cartilage defects to a certain extent, the larger trauma can easily lead to complications in the donor area.

Tissue engineering is an interdisciplinary subject involving cell biology, material science, engineering and bioreactor. It uses the basic principles and methods of life science and engineering to build the tissues needed by the human body to repair and replace the tissues or organs that have no function due to trauma and disease. In recent years, with the progress of tissue engineering, people have gradually begun to study the use of scaffolds or tissues constructed by tissue engineering to try to repair cartilage defects. By combining with medical degradable materials, such as decalcified bone materials, the construction of cartilage tissue is expected to become a new method for tissue engineering cartilage construction. However, the decalcified bone matrix has a large pore size and good porosity, but the cell adhesion rate is extremely low when inoculated with chondrocyte suspension, which is not conducive to the construction of tissue engineering materials.

In summary, there is an urgent need to develop a method for cartilage regeneration by inoculating chondrocytes into a framework structure to form a tissue engineered cartilage composites in this field.

### SUMMARY OF THE INVENTION

The purpose of the present invention is to provide a method for realizing cartilage regeneration by inoculating chondrocytes into a framework structure.

In the first aspect of the present invention, it provides a gel cartilage, which comprises a cell population of chondrocytes and extracellular matrix secreted by chondrocytes, wherein the extracellular matrix encapsulates the cell population, and the gel cartilage is in a gel state, and the density of chondrocytes is at least 1.0×10⁸ cells/ml or 1.0×10⁸ cells/g.

In another preferred embodiment, the gel cartilage is prepared by gelation culture of chondrocytes.

In another preferred embodiment, the adhesion rate of the gel cartilage is ≥90%, preferably ≥95%.

In another preferred embodiment, in the gel cartilage, the concentration of chondrocytes is 1.0×10⁸ cells/ml-10×10⁸ cells/ml, preferably 1.5-5×10⁸ cells/ml.

In another preferred embodiment, the gel cartilage is obtained by gelation culture for 2-5 days, preferably for 2.5-4 days.

In another preferred embodiment, the chondrocytes are derived from a mammal.

In another preferred embodiment, the chondrocytes are selected from elastic cartilage, fibrocartilage or hyaline cartilage.

In another preferred embodiment, the chondrocytes are selected from ear chondrocytes, costal cartilage, or a combination thereof.

In another preferred embodiment, the ear chondrocytes are derived from the autologous or allogeneic sources, preferably autologous ear chondrocytes.

In the second aspect of the present invention, it provides a tissue engineering cartilage complex, which comprises:
(a) a carrier comprising a porous biocompatible materials; and
(b) gel cartilage of the first aspect of the present invention that are inoculated on or loaded on the carrier.

In another preferred embodiment, the complex comprises a complex formed by inoculating the gel cartilage on the carrier and undergoing chondrogenic culture (in the complex, the chondrocytes are supported on the carrier and form a more cohesive integrated structure with the carrier).

In another preferred embodiment, the complex comprises a complex formed by inoculating the gel cartilage on the carrier without chondrogenic culture.

In another preferred embodiment, the ratio of gel cartilage to porous biocompatible material (or carrier) in the complex is 0.1-0.2 ml (or g) for gel cartilage: 0.5-1 g for porous biocompatible material.

In another preferred embodiment, the porosity of the porous biocompatible material is ≥30%, preferably ≥50%, more preferably ≥70%.

In another preferred embodiment, the porosity of the porous biocompatible material is 80%-95%.

In another preferred embodiment, the porous biocompatible material has an aperture of 400-800 μm.

In another preferred embodiment, the porous biocompatible material includes a biodegradable material.

In another preferred embodiment, the biodegradable material is selected from the group consisting of PCL, PGA, allogeneic bone repair material, xenogeneic bone repair material, and decalcified bone matrix.

In another preferred embodiment, the biodegradable material may further be loaded with gelatin, collagen, silk fibroin, hydrogel, or a combination thereof.

In another preferred embodiment, the biodegradable material is a decalcified bone matrix.

In another preferred embodiment, the decalcified bone matrix is derived from allogeneic or xenogeneic bone.

In another preferred embodiment, the tissue engineering cartilage complex is prepared by the method of the fourth aspect of the present invention.

In the third aspect of the present invention, it provides a method for preparing the gel cartilage of the first aspect of the present invention, which comprises the following steps:
(1) Providing isolated chondrocytes for primary culture and subculture to obtain subcultured chondrocytes;
(2) The subcultured chondrocytes obtained in step (1) being induced and cultured in gelation culture medium to obtain induced gel cartilage.

In another preferred embodiment, in step (2), the subcultured chondrocytes are inoculated in a culture container in a layered manner.

In another preferred embodiment, in step (2), the number of cells S1 of the laminated inoculation of the present invention is n times of the number of cells S0 for the degree of confluence of 100% (i.e., S1/S0=n), wherein n is 1.5-20, preferably 2-10, more preferably 2.5-5.

In another preferred embodiment, in step (1), the concentration (m/v) of the collagenase is 0.1%-0.2%.

In another preferred embodiment, in step (1), the time for the collagenase digestion is 6-10 hours.

In another preferred embodiment, the chondrocytes are subcultured to passage 2-5.

In another preferred embodiment, in step (2), the inoculation density of the chondrocytes is 0.5×10⁶-3×10⁷ cells/cm²; preferably, 1×10⁶-5×10⁶ cells/cm²; more preferably, 1×10⁶-3×10⁶ cell s/cm².

In another preferred embodiment, in step (2), the culture medium contains or does not contain serum.

In another preferred embodiment, in step (2), the culture medium contains 5-15% (v/v) of serum.

In another preferred embodiment, the serum is selected from fetal bovine serum.

In another preferred embodiment, in step (2), the gelation medium is DMEM medium.

In another preferred embodiment, in step (2), the DMEM medium further contains 4-5 wt % glucose, 5-20% FBS (v/v), 50-150 U/ml penicillin-streptomycin.

In another preferred embodiment, in step (2), the time for the induction culture is 2-5 days, preferably 2.5-4 days.

In the fourth aspect of the present invention, it provides a method for preparing the tissue engineering cartilage complex of the third aspect of the present invention, comprising the steps of: inoculating the gel cartilage of the first aspect of the present invention into a porous biocompatible material, and performing in vitro chondrogenic culture, thereby obtaining the tissue engineering cartilage complex.

In another preferred embodiment, the time for chondrogenic culture is 2-15 days, preferably 3-11 days, more preferably 4-7 days.

In the fifth aspect of the present invention, it provides a use of the tissue engineering cartilage complex of the second aspect of the present invention for preparing a medical product for repairing cartilage and/or hard tissue defects.

In another preferred embodiment, the cartilage and/or hard tissue defect is selected from: joint cartilage defect, cleft lip and palate deformity, maxillofacial hard tissue defect, or a combination thereof.

In another preferred embodiment, the tissue engineering cartilage complex comprises a tissue engineering cartilage graft.

In another preferred embodiment, the shape of the tissue engineering cartilage graft corresponds to the shape of the defect site in the human body where cartilage needs to be transplanted.

In another preferred embodiment, the defect site is selected from: joint cartilage defect, cleft lip and palate deformity, maxillofacial hard tissue, or a combination thereof.

In another preferred embodiment, the shape of the tissue engineering cartilage graft includes various shapes such as human auricle, nasal dorsum, nasal alar, zygomatic arch, eyebrow arch, tubular, rhombic, flaky, cylindrical, etc., but is not limited to this.

In the sixth aspect of the present invention, it provides a method for repairing cartilage and/or hard tissue defects related diseases, characterized in that the tissue engineering cartilage complex of the second aspect of the present invention is administrated to a subject in need thereof.

In another preferred embodiment, the cartilage and/or hard tissue defect is selected from: joint cartilage defect, cleft lip and palate deformity, or maxillofacial hard tissue defect.

It should be understood that within the scope of the present invention, each technical features of the present invention described above and in the following (such as examples) may be combined with each other to form a new or preferred technical solution, which is not listed here due to space limitations.

### DESCRIPTION OF THE DRAWINGS

FIG. 1 shows the decalcified bone matrix frame.

FIG. 2 shows the electron microscope of the decalcified bone matrix frame, and the bar is 1 mm.

FIG. 3 shows a schematic of the culture of gel cartilage. Among them, A is the cartilage gel cultured for 3 days in a six-well plate, B is the cartilage gel at the bottom of the six-well plate after absorbing the medium at the middle and upper part of the six-well plate, and C is the injectable preparation containing cartilage gel after adding appropriate medium to the cartilage gel; D is the cartilage sheet cultured in a six-well plate for 15 days, and E is a schematic diagram of the cartilage sheet with a certain mechanical strength; F is an injectable preparation containing cartilage sheet prepared after cutting the cartilage sheet.

FIG. 4 shows an injectable preparation containing cartilage gel (cultured for 3 days).

FIG. 5 shows the demineralized bone matrix framework inoculated with gel cartilage preparation, and the integrated tissue engineered cartilage prepared after adding chondrogenic culture medium for continuous culture.

FIG. 6 shows the electron microscope image of the prepared integrated tissue engineered cartilage at a scale of 200 μm.

FIG. 7 shows a comparison of the adhesion rate of the inoculated sample (cell suspension or cartilage gel) after culture on the complex for 24 hours.

FIG. 8 shows a comparison of gel cartilage-decalcified bone complex (A) and simple decalcified bone (B) transplanted to the knee joint defect site in a goat.

### DETAILED DESCRIPTION

Through extensive and in-depth research, the inventor unexpectedly discovered for the first time that a novel gel-like cartilage can be formed by inoculating and/or spreading a specific number of chondrocytes on a flat or basically flat culture surface, so that the inoculated chondrocytes forming a specific laminated structure, and culturing the laminated chondrocytes under suitable gelation culture conditions. The gel-like cartilage of the present invention has the advantages of strong adhesion, easy inoculation on the solid carrier to form a composite, high degree of composite with the carrier in the later chondrogenic culture, immediate repair ability of the cartilage graft, excellent cartilage repair effect, etc. The present invention has been completed on this basis.

In addition, the inventor also developed a complex or graft based on the gel cartilage of the present invention, that is, the gel cartilage of the present invention is inoculated into a porous carrier material (also known as a “frame structure”) to form a complex for cartilage regeneration.

By optimizing the culture medium for in vitro culture and the time of gel culture, compared with ordinary chondrocytes, the particle size of the gel cartilage in the present invention is significantly increased, which is conducive to the attachment of decalcified bone matrix material, avoids the loss of chondrocytes, and significantly reduces the pore size ratio of the tissue engineering bone graft. The tissue engineering bone graft of the present invention has an immediate repair effect on joint defects, and the microenvironment of gel cartilage is conducive to the formation of mature cartilage tissue at joint defects. Therefore, it is beneficial for achieving cartilage regeneration and is used for various filling treatments and bone repair. The present invention has been completed on this basis.

### Term

As used herein, “tissue engineering complex of the present invention”, “gel cartilage tissue engineering complex” and “tissue cartilage graft of the present invention” can be interchangeably used, all of which refer to the tissue engineering cartilage complex of the second aspect of the present invention.

As used herein, the term “gelation culture” refers to the process of providing a special biochemical environment to enable cells with cartilage differentiation potential to express and form gel-like chondrocytes with thick texture and significantly increased particle size, which are capable of chondrogenesis.

As used herein, the term “cartilage (stem) cells” refers to chondrocytes, cartilage stem cells, or a combination thereof.

Cartilage Gel and Preparation Thereof

As used herein, “gel cartilage”, “cartilage gel”, “gel-state cartilage”, “gel-like cartilage”, “cartilage gel of the present invention” or “gel cartilage of the present invention” can be used interchangeably, all refer to the cartilage (stem) cells in gel state of the present invention, in particular, chondrocytes with a specific concentration are inoculated on and/or spread on a flat or substantially flat culture surface, so that the inoculated chondrocytes form a laminated structure, and the chondrocytes having a laminated structure are cultured under suitable gelation culture conditions, thereby forming a gel-like cartilage culture.

The gel cartilage of the present invention is a new type of cartilage different from free chondrocytes, centrifugally precipitated chondrocytes and cartilage pellet. The gel cartilage of the present invention can be regarded as a specific form of cartilage between free chondrocytes and dense cartilage masses. During the process of gelation culture of the gel cartilage of the present invention, the chondrocytes not only contact and/or interact with adjacent cells on the plane (X-Y plane), but also contact and/or interact with adjacent chondrocytes in multiple directions such as above and/or below and/or the upper or lower side, so as to promote the chondrocytes to secrete and form more extracellular matrix, therefore, the gelation cultured chondrocytes are wrapped in extracellular matrix with a certain viscosity, so that the gel cartilage of the present invention has a close connection, but has a certain viscosity and fluidity. Therefore, the gel cartilage of the present invention is more suitable for inoculation and loading on various carrier materials (especially porous carrier materials), which can forma complex for repairing cartilage.

In addition, the gel cartilage of the present invention has a gel state on the one hand, and on the other hand, it has an unusually high cell density (usually at least 1.0×10⁸ cells/ml or more, such as 1.0×10⁸-10×10⁸ cells/ml). Therefore, it is particularly suitable for the preparation of grafts for repairing various types of cartilage, or for cartilage transplantation or cartilage repair surgery.

In the present invention, the complex for repairing cartilage includes the complex formed by loading the gel cartilage of the present invention on a carrier material (especially a porous biocompatible material) without undergoing chondrogenic culture, and also includes the complex formed by loading the gel cartilage of the present invention on a carrier material (especially a porous biocompatible material) and undergoing chondrogenic culture.

In the present invention, the complex suitable for transplantation to a human or animal body is the tissue engineering cartilage complex of the present invention.

Preferably, in the present invention, the gel cartilage is formed by in vitro culture for a period of time t1 under the gelation culture condition. Preferably, the t1 is 2.5-5.5 days, preferably 3-5 days.

In the present invention, one feature is laminated inoculation, that is, after chondrocytes with a specific density are inoculated into a culture container, the inoculated chondrocytes will form a multilayer chondrocyte group (i.e., a chondrocyte group with a laminated structure) through, for example, deposition. Typically, calculated on the basis of the culture area of the culture dish (or culture container), and assuming that the degree of confluence of the monolayer cells is 100%, the number of cells S1 of the laminated inoculation of the present invention is n times of the number of cells S0 for the degree of confluence of 100% (i.e., S1/S0=n), wherein n is 1.5-20, preferably 2-10, more preferably 2.5-5.

In a specific embodiment, chondrocytes with a high concentration are inoculated on a culture medium with a smooth surface. Through the gelation culture medium in the present invention, the chondrocytes are induced in vitro for 2-5 days, forming a state between free and close contact, that is, a “quasi contact” cell state. Specifically, the chondrocytes of the present invention are cultured in vitro and wrapped in several chondrocytes through an extracellular matrix, forming a structure with relatively close cell contact.

As used herein, the “chondrocytes with a high concentration” refers to inoculating 1.0×10⁷-2.0×10⁷ cells, preferably 1.5×10⁷ cells, into a 3.5 cm culture dish (e.g., one well in a six-well plate).

In the gel cartilage, the concentration of cells is 1.0×10⁸ cells/ml-10×10⁸ cells/ml, preferably 1.5-5×10⁸ cells/ml.

Specifically, the gel cartilage of the present invention has a certain adhesion rate. Through the adhesion rate measurement method in a preferred example of the present invention, the adhesion rate of the gel cartilage in the present invention is ≥90%, preferably ≥95%.

The gel cartilage of the present invention is differentiated from high-density cartilage gel cells. The high-density chondrocytes of the present invention grow into gel cartilage through in vitro culture. The particle size of gel cartilage is significantly increased, which is more conducive to the attachment of decalcified bone matrix. The concentration range of chondrocytes is preferably 1.6×10⁶-2.2×10⁶ cells/cm².

In another preferred embodiment, the gelation culture condition is: inoculating chondrocytes with a high density, and using high-glucose medium containing 10% fetal bovine serum and 1% three antibiotics for culture.

In another preferred embodiment, the time for gel cartilage culture is 2-5 days, and the inoculation density is 1.6×10⁶-2.2×10⁶ cells/cm².

The present invention provides a specific gel material, namely gel-like chondrocytes, which is cultured for 3 days.

Decalcified Bone Matrix

Decalcified bone matrix (DBM) is a bone graft material that can reduce immunogenicity by decalcification of allogeneic bone or xenogeneic bone. Different degrees of decalcification correspond to different mechanical strengths. It has good biological characteristics, osteoinductivity, osteoconductivity and biodegradability, promotes new bone formation and bone tissue mineralization, and then accelerates bone healing. It can effectively repair bone damage alone or in combination with autologous bone, other biological materials and growth factors. It is an ideal bone tissue engineering scaffold material. However, the general decalcified bone matrix has a large pore size and extremely low cell adhesion rate when inoculated with chondrocyte suspension, which is not conducive to the construction of tissue engineering carriers.

In another preferred embodiment, the decalcified bone matrix of the present invention has a pore size of 400-800 μm and a porosity of 87.3%±3.7%.

Tissue Engineering Cartilage Graft

Simple gel cartilage cannot be molded. Under tension conditions, the absorption rate of simple gel cartilage is high, and clinical application is limited. Using decalcified bone matrix as a frame structure, a special shape gel cartilage decalcified bone matrix complex can be constructed, and the absorption rate of cartilage can be limited after providing mechanical support.

It should be understood that in addition to the decalcified bone matrix used in specific embodiments, the gel cartilage in the present invention can also be loaded on porous materials of other common organisms after being inoculated on the decalcified bone, preferably on degradable materials, including but not limited to:
(a) biodegradable synthetic polymer materials, such as polylactic acid (PLA), polyhydroxyacetic acid (PGA), PLGA, polyhydroxybutyric acid (PHB), polyanhydrides, polyphosphazenes, polyamino acid, pesudo-polyamino acid, polyoxoesters, polyethylene glycol, hyaluronic acid, polydioxanone, etc;
(b) natural degradable materials, such as collagen, gelatin, glycosaminoglycans (GAGs), chitosan, chitin, alginate, and various acellular matrices such as decalcified bone matrix;
(c) copolymers or composite materials of the above materials, especially composite materials of polymer materials and natural materials, as well as composite materials of solid materials and injectable materials.

The preferred medically acceptable biodegradable materials are solid materials or solid/liquid composite materials, such as polylactic acid (PLA), polylactic acid (PGA), collagen, decalcified bone matrix, etc. The materials in the present invention can be prefabricated into various precise sizes and shapes to adapt to the construction of cartilage tissues of different sizes and shapes. When the material is a solid material, it can be directly prefabricated into the required size and shape, or precise plasticity can be achieved through computer assisted and rapid prototyping models.

Preparation Method

The preparation method of the tissue engineering cartilage graft of the present invention is simple, and the method comprises the steps of:
(1) culture of chondrocytes;
(2) induction culture of gel cartilage;
(3) inoculation of tissue engineering carriers.

Adhesion Rate Measurement Method

The specific steps of the adhesion rate measurement method in the present invention are as follows:

Detect the DNA quantification A1 of inoculated samples (e.g., cell suspension or cartilage gel); Detect the DNA quantification A2 of the post-inoculation complex (e.g., cell-frame complex or cartilage gel-frame complex) after 24 hours of culture. The adhesion rate is A2/A1×100%.

The determination method for DNA quantification includes the following steps: Taking inoculated samples (such as cartilage gel or cartilage gel-frame complex) and digesting them with protease K. The digested samples are quantitatively detected using PicoGreen kits (Invitrogen, Carlsbad, CA, USA), absorbance of 520 nm is determined using fluorescent microplate reader, and DNA content is calculated according to standard curve formula.

The Main Advantages of the Present Invention Include:
(1) Gel cartilage tissue is more mature than chondrocytes and has certain fluidity.
(2) Decalcified bone matrix, as a biodegradable natural material, can be degraded in the body, resulting in a low immune response and good biological safety.
(3) The decalcified bone matrix material has a large pore size and good porosity. The gel cartilage tissue with certain fluidity and relatively thick can effectively improve the adhesion rate. (4) Simple gel cartilage cannot be molded. Under tension conditions, the absorption rate of simple gel cartilage is high, and clinical application is limited. Decalcified bone matrix can be used to construct gel cartilage decalcified bone matrix complex, and it can provide mechanical support, and reduce the absorption rate of cartilage.
(5) According to the demand, the decalcified bone is customized to different shapes, and the gel cartilage decalcified bone complex can be constructed for repair and reconstruction of different parts and shapes.

The present invention is further explained below in conjunction with specific example. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

Culture Medium

Chondrogenic medium: high glucose DMEM medium, 1% 1×ITS premium (ITS universal culture mixture, containing insulin, transferrin, selenite, linoleic acid, bovine serum protein, pyruvate, ascorbic acid phosphate), 40 μg/ml proline, 10 ng/ml TGF-01, 100 ng/ml IGF-1, 40 ng/ml dexamethasone and 50 μg/ml vitamin C.

Gelation medium: DMEM medium containing 4-5 wt % glucose, 10% FBS (v/v) and 100 U/ml penicillin-streptomycin.

### Example 1: In Vitro Culture of Gel Cartilage

2.5×2.5 cm² ear cartilage tissue was aseptically cut, and sterile instruments were used to peel off the mucosa and fibrous tissue on the cartilage surface. The cartilage tissue was cut into 1.5×1.5 mm² sized cartilage pieces. A concentration of 0.15% collagenase was prepared; and the cartilage pieces were added to the prepared collagenase for digestion for 8 hours. After 8 hours, collagenase solution was filtered and centrifuged to obtain ear chondrocytes, and primary and subculture were conducted. The cells were subcultured to passage 2-5, preferably passage 3.

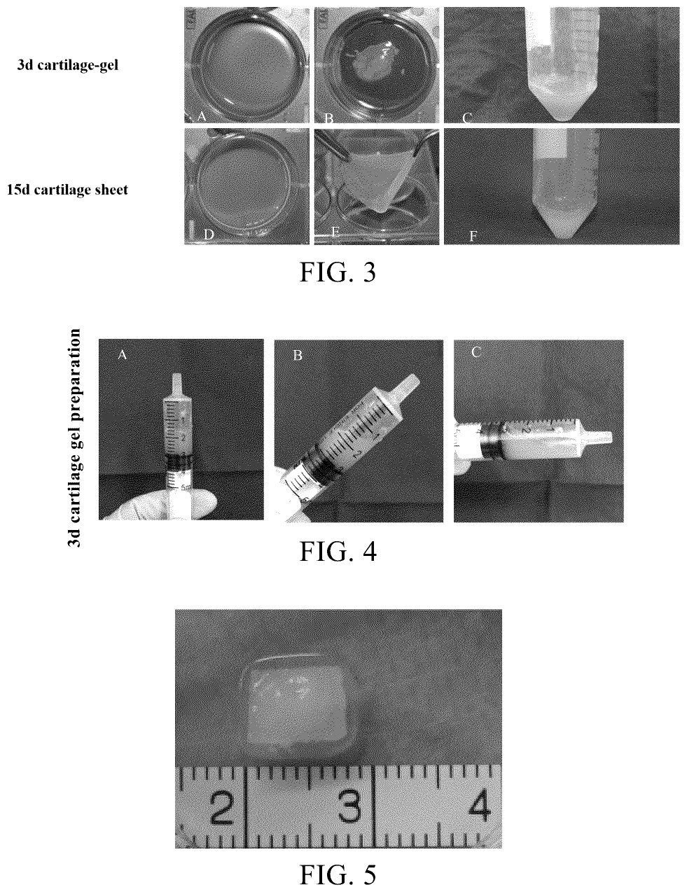

After expansion, the cells were collected and resuspended, and the cells were inoculated into a six-well plate (3.5 cm in diameter) with the cell volume of 8×10⁶ cells/10 ml to 30×10⁶ cells/10 ml/well, and cultured in gelation medium (DMEM medium containing 4-5 wt % glucose, 10% FBS (v/v) and 100 U/ml penicillin-streptomycin). After 3 days of culture, the medium in the middle and upper part of the six-well plate was sucked off, and the cartilage gel at the bottom of the six-well plate was gathered with tweezers (see FIG. 3B). The output of gel cartilage in one well was 0.1-0.2 ml, and collected into a 5 ml syringe.

After 3 days, the cartilage gel obtained is about 0.1 ml, and the cell density in the cartilage of the gel is 1.0×10⁸ cells/ml-10×10⁸ cells/ml, preferably 1.5-5×10⁸ cells/ml.

It was mixed with 0.15 ml of medium to make an injectable preparation containing cartilage gel, as shown in FIG. 3C, FIG. 4.

As shown in FIG. 3, on the third day, the viscosity of cartilage gel was significantly improved, which was easy to combine with load materials with large pore size.

In particular, when the cartilage gel of the third day was cultured to the 15th day, a lamellar cartilage sheet was obtained, which was thin and had good mechanical strength.

### Example 2: Construction of Gel Cartilage Tissue In Vitro

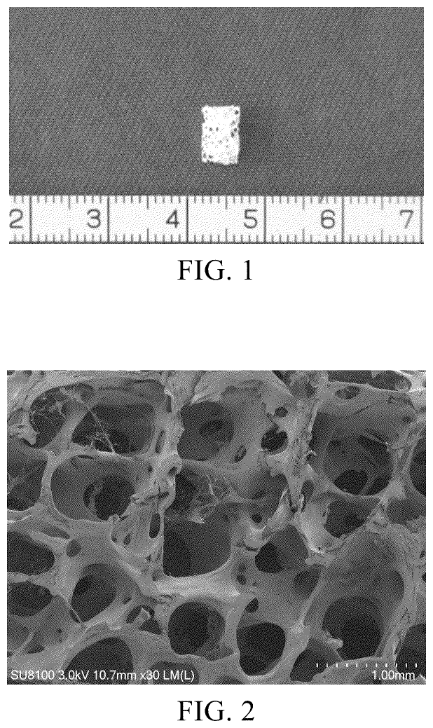

A decalcified bone matrix framework (as shown in FIG. 1) was provided, which has been determined to have approximately 400-800 μm pore size, with a porosity of approximately 87.3%±3.7% (the electron microscopy observation results of the decalcified bone matrix framework are shown in FIG. 2).

The gel cartilage preparation (prepared in Example 1, with a volume of about 0.25-0.35 ml) was inoculated into the decalcified bone matrix framework, and placed at 37° C., 95% humidity, and 5% carbon dioxide for 2 hours. Then chondrogenic medium was added for further cultivation for 3-11 days to obtain integrated tissue engineered cartilage (FIG. 5).

Results

The overall structure of the integrated tissue engineered cartilage prepared is shown in FIG. 5.

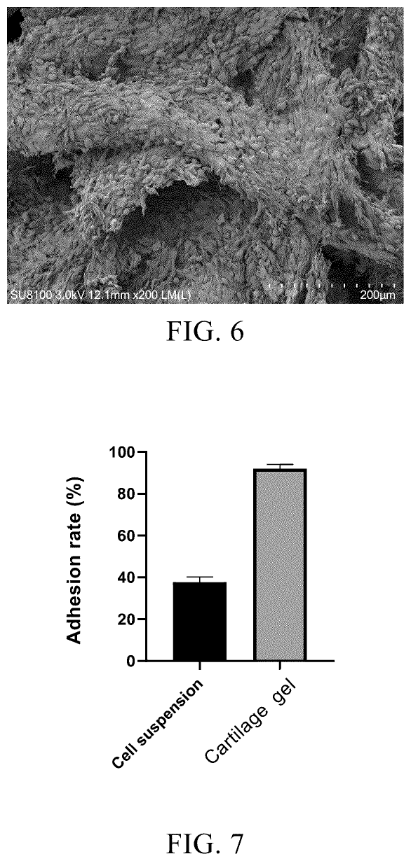

The electron microscopic observation results are shown in FIG. 6, which shows that the pores of decalcified bone matrix are basically effectively filled after the inoculation of gel cartilage.

### Example 3: Determination of Adhesion Rate

A decalcified bone matrix framework was provided (as shown in FIG. 1). The gel cartilage preparation (prepared in Example 1, with a volume of about 0.25-0.35 ml) was inoculated into the above-mentioned decalcified bone matrix framework, and the cells were placed at 37° C., 95% humidity, and 5% carbon dioxide for 2 hours, and subcultured for 4 times.

As shown in FIG. 7, the adhesion rate is calculated using the adhesion rate measurement method described above. Compared with the cell suspension, the adhesion rate of the gel cartilage of present the invention is 92%±2%.

### Example 4: Animal Transplantation Experiment for Repairing Articular Cartilage

The gel cartilage was inoculated into decalcified bone to construct the gel cartilage decalcified bone complex, and was continuously cultured in vitro for 11 days.

A cartilage defect with a diameter of 7.5 mm was made on the articular surface of the knee joint of the experimental animal, and the gel cartilage-decalcified bone complex was used to repair the defect.

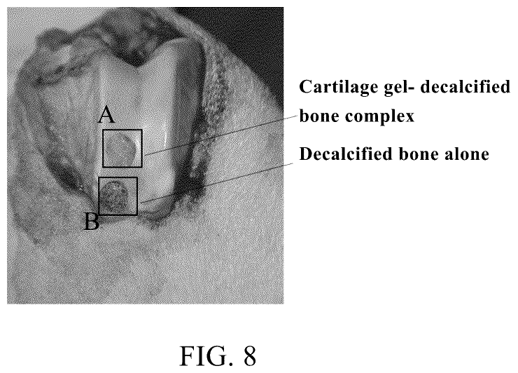

As shown in FIG. 8, the defect is filled by gel cartilage-decalcified bone complex at position A, and the defect is filled by pure decalcified bone at position B.

Observation of the wound surface; The defect area at position A is smooth and solid, surrounded by a soft tissue membrane, with a certain degree of elasticity and immediate functional repair effect.

The defect area at position B is rough and has only physical support, which cannot be repaired immediately.

The experimental results show that by using the gel cartilage-decalcified bone complex to repair articular cartilage, the structure of the tissue was similar to that of normal cartilage tissue, which can play an immediate repair effect on joint defects and help to form mature cartilage tissue at joint defects.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A gel cartilage, which comprises a cell population of chondrocytes and extracellular matrix secreted by chondrocytes, wherein the extracellular matrix encapsulates the cell population, and the gel cartilage is in a gel state, and the density of chondrocytes is at least 1.0×10⁸ cells/ml or 1.0×10⁸ cells/g.

2. The gel cartilage of claim 1, which is prepared by gelation culture of chondrocytes.

3. The gel cartilage of claim 1, wherein the adhesion rate of the gel cartilage is ≥90%.

4. The gel cartilage of claim 1, wherein the concentration of chondrocytes in the gel cartilage is 1.0×10⁸ cells/ml-10×10⁸ cells/ml, preferably 1.5-5×10⁸ cells/ml.

5. The gel cartilage of claim 1, wherein the gel cartilage is obtained by gelation culture for 2-5 days, preferably for 2.5-4 days.

6. The gel cartilage of claim 1, wherein the chondrocytes are selected from elastic cartilage, fibrocartilage, or hyaline cartilage.

7. A tissue engineering cartilage complex, which comprises:
(a) a carrier comprising a porous biocompatible materials; and
(b) the gel cartilage of claim 1 that are inoculated on or loaded on the carrier.

8. The tissue engineering cartilage complex of claim 7, wherein the porosity of the porous biocompatible material is ≥30%, preferably ≥50%, more preferably ≥70%.

9. The tissue engineering cartilage complex of claim 7, wherein the pore size of the porous biocompatible material is 400-800 μm.

10. The tissue engineering cartilage complex of claim 7, wherein the porous biocompatible material comprises a biodegradable material.

11. The tissue engineering cartilage complex of claim 10, wherein the biodegradable material is selected from the group consisting of PCL, PGA, allogeneic bone repair material, xenogeneic bone repair material, or decalcified bone matrix.

12. A method for preparing the tissue engineering cartilage complex of claim 1, which comprises the steps:
(1) Providing isolated chondrocytes for primary culture and subculture to obtain subcultured chondrocytes;
(2) The subcultured chondrocytes obtained in step (1) being induced and cultured in gelation culture medium to obtain induced gel cartilage.

13. The method of claim 12, in step (2), the subcultured chondrocytes are inoculated in a culture container in a layered manner.

14. A method for preparing the tissue engineering cartilage complex of claim 7, comprising the steps of: inoculating a gel cartilage into a porous biocompatible material, and performing in vitro chondrogenic culture, thereby obtaining the tissue engineering cartilage complex,
wherein the gel cartilage comprises a cell population of chondrocytes and extracellular matrix secreted by chondrocytes, wherein the extracellular matrix encapsulates the cell population, and the gel cartilage is in a gel state, and the density of chondrocytes is at least 1.0×10⁸ cells/ml or 1.0×10⁸ cells/g.

15. Use of the tissue engineering cartilage complex of claim 7 for preparing a medical product for repairing cartilage and/or hard tissue defects.

16. A method for repairing cartilage and/or hard tissue defects related diseases, by administrating the tissue engineering cartilage complex of claim 7 to a subject in need thereof.

17. The method of claim 16, wherein the cartilage and/or hard tissue defect is selected from: joint cartilage defect, cleft lip and palate deformity, or maxillofacial hard tissue defect.
